(19) United States
(12) Patent Application Publication
(10) Pub. No.: US 2005/0262597 A1
(43) Pub. Date: Nov. 24, 2005

(54) Method for increasing transgene expression

(75) Inventors: Willem Broekaert, Dilbeek (BE); Chris DeWilde, Herdersem-Aalst (BE); Yves Hatzfeld, Lille (FR); Zhongyi Zhou, Balegem (BE)

Correspondence Address: DILWORTH & BARRESE, LLP, 333 EARLE OVINGTON BLVD., UNIONDALE, NY, 11553, US

(73) Assignee: CropDesign, N.V., Gent (BE)

(21) Appl. No.: 11/128,549

(22) Filed: May 13, 2005

Related U.S. Application Data
(60) Provisional application No. 60/572,141, filed on May 18, 2004.

(30) Foreign Application Priority Data
May 13, 2004 (EP) EP04102108.0

(51) Int. Cl. A01H 1/00; C12N 15/82; A01H 5/00

(52) U.S. Cl. 800/287; 800/320.1; 800/320.2

(57) ABSTRACT

The present invention provides a method for increasing expression of a transgene using the 5′ untranslated region (5′UTR) of a GOS2 gene, or part of such 5′UTR, comprising the first intron. The 5′UTR is integrated in or at the 5′ end of a nucleic acid of interest and is combined with a plant expressible promoter. The nucleic acid provided may be used in methods for modifying growth characteristics of transgenic plants relative to corresponding wild type plants.

### CROSS REFERENCE TO RELATED APPLICATIONS

This application claims the benefit of U.S. Provisional Application Ser. No. 60/572,141 filed May 18, 2004, which application is incorporated by reference herein.

### BACKGROUND OF THE INVENTION

1. Technical Field

The present invention relates to methods for increasing transgene expression in plants. In particular, the present invention relates to the use of the 5′ untranslated region (5′UTR) of a GOS2 gene, particularly to the use of the first intron of a GOS2 gene for enhancing transcription. This invention also relates to methods for modifying the composition of seeds and in particular for increasing transcription or protein expression in plants.

2. Description of the Related Art

Protein levels in a cell are determined on the one hand by their rate of degradation and on the other hand by their rate of synthesis. The rate of protein synthesis is dependent on various processes, such as transcription, post-transcriptional RNA processing, translation and post-translational modifications. The first step in protein synthesis is transcription, the process of copying DNA into an RNA molecule. Besides the promoter, which is the most important part of a gene controlling the level and pattern of transcription, it is also well known that the 5′ untranslated region (5′UTR) of a primary RNA transcript has influence on the level and pattern of expression of plant genes. Several plant genes are known to contain an intron in their 5′UTR; gene expression and regulation studies have demonstrated that these introns may have a positive impact on transcription. Examples include introns from Arabidopsis (Rose and Last, Plant J. 11, 455-464, 1997; Chaubet-Gigot et al., Plant Mol. Biol. 45, 17-30, 2001), castor bean (Ricinus communis; Tanaka et al., Nucleic Acids Res. 18, 6767-6770, 1990), oat (Avena sativa; Bruce and Quail, Plant Cell 2, 1081-1089, 1990), petunia (Petunia hybrida; Dean et al., Plant Cell 1, 201-208, 1989; Vain et al., Plant Cell Rep. 15, 489-494, 1996), rice (Oryza sativa; McElroy et al., Plant Cell 2, 163-171, 1990; Snowden et al., Plant Mol. Biol. 31, 689-692, 1996; Rethmeier et al., Plant J. 12, 895-899, 1997), soybean (Glycine max; Kato et al., Biosci. Biotechnol. Biochem. 62, 151-153, 1998), potato (Solanum tuberosum; Leon et al., Plant Physiol. 95, 968-972, 1991; Fu et al., Plant Cell 7, 1387-1394, 1995; Fu et al., Plant Cell 7, 1395-1403, 1995), and tobacco (Nicotiana tabacum; Plesse et al., Plant Mol. Biol. 45, 655-667, 2001).

The mechanism underlying this intron-mediated enhancement of transcription is still unclear, but the introns must be in their normal orientation with respect to the transcribed region of the gene (Callis et al., Genes Dev. 1, 1183-1200, 1987; Clancy et al., Plant Sci. 98, 151-161, 1994; Mascarenhas et al., Plant Mol. Biol. 15, 913-920, 1990). A study of PATI intron 1 in Arabidopsis (Rose, RNA 8, 1444-1453, 2002) demonstrated that the splicing machinery was required, but that intron splicing was not enough to enhance mRNA accumulation. According to the model presented, introns could stimulate mRNA accumulation by increasing the processing capacity of RNA polymerase 11 via a modification of its carboxy-terminal domain, thereby promoting transcript elongation without significantly affecting transcript initiation. The transcription of genes containing an intron would therefore be more likely to extend the length of the gene in cases where 3′ end processing would produce stable transcripts. Studies suggest that the presence of an intron increases the probability of full-length stable transcripts being made, thereby leading to increased mRNA accumulation (Rose, 2002). Clancy and Hannah (Plant Physiol. 130, 918-929, 2002) used reporter gene fusions to identify features of the Sh1 first intron required for enhancement in cultured maize cells. They found that a 145-bp derivative of the Sh1 intron conferred approximately the same 20- to 50-fold stimulation typical for the full-length intron in their transient expression system. Furthermore, a 35-bp motif contained within the intron was found to be required for maximum levels of enhancement but not for efficient transcript splicing. The important feature of this redundant 35-bp motif is T-richness rather than the specific sequence. When transcript splicing was abolished by mutations at the intron borders, enhancement was reduced to about 2-fold. The requirement of splicing for enhancement was found not to be due to upstream translation initiation codons contained in unspliced transcripts. The authors concluded that splicing of the Sh1 intron was integral to enhancement and suggested that transcript modifications triggered by the T-rich motif and splicing may link the mRNA with the trafficking system of the cell. Other reports suggest that length and composition of flanking sequences may contribute to intron-mediated enhanced gene expression (Sinibaldi and Mettler, In W E Cohn, K Moldave, eds, Progress in Nucleic Acid Research and Molecular Biology, Vol. 42. Academic Press, New York, pp 229-257, 1992; Luehrsen and Walbot, Prog. Nucleic Acid Res. Mol. Biol. 47, 149-193, 1991; Maas et al., Plant Mol. Biol. 16, 199-207, 1991; Clancy et al., 1994), or that the magnitude of stimulation also depends on the coding sequences (Sinibaldi and Mettler, 1992; Rethmeier et al., 1997; Rethmeier et al., Plant J. 13, 831-835, 1998), the tissue of expression, and physiological conditions (Tanaka et al., 1990; Sinibaldi and Mettler, 1992; Gallie and Young, Plant Physiol. 106, 929-939, 1994; Fu and Park, 1995a, 1995b; Chaubet-Gigot et al., 2001; Plesse et al., 2001). GOS2 from rice is a single copy gene which encodes the translation initiation factor eIF1, which is involved in the assembly of the 43S complex with mRNA and in the correct scanning of the 5′UTR towards the ATG during initiation of translation. The GOS2 gene is also found in other organisms such as yeast and mammals, and for each of these, the first intron is located in the 5′UTR. In its natural environment, the rice GOS2 protein is expressed under control of its strong and constitutive promoter. This promoter is widely used as an alternative for the CaMV35S promoter in the genetic engineering of monocotyledonous plants. Hensgens et al. (Plant Mol. Biol. 23, 643-669, 1993) studied expression of the reporter gene gusA driven by the GOS2 promoter. They describe a first type of construct comprising, from 5′ to 3′ a GOS2 transcriptional promoter, the translation initiation site, the first two exons and introns and part of the third exon, a polylinker region, the gusA coding sequence and a polyadenylation region. Hensgens et al. also describe a second type of construct, in which gusA was fused to the first 36 nucleotides of the first GOS2 exon and thus lacked the first two introns. The highest GUS expression was observed for the first type of construct, however the authors could not determine whether the difference in expression level between the two types of constructs was attributable to the presence of the two introns. To date, no analysis has been performed of the role of the introns in the GOS2 gene in the process of transcription.

Plant phenotypic properties are determined by expression of one or more genes in the plant genome. The appropriate expression level and pattern (temporal and spatial expression patterns) of genes may depend on many regulatory elements, including the type of promoter and the untranslated regions of the mRNA. These regulatory elements may advantageously be used in adapting expression of genes to particular needs. For example, a promoter may have the desired spatial expression (such as root-specific or flower-specific expression), but may lack the strength to ensure high expression levels. In such cases, a person skilled in the art may consider using additional regulatory elements to enhance expression levels. Examples of regulatory elements for modulating expression levels or patterns are known in the art and include upstream activating sequences, promoter-proximal elements, enhancers and silencers. A number of genes have been described for which the presence of introns contributed significantly to the expression level. However the mechanism of this increase is poorly understood.

In order to obtain desired gene expression levels or patterns, which may be fine-tuned depending on specific needs, it is desirable to have at one's disposal a wide array of introns that are capable of stimulating gene expression. A need exists in various fields of genetic engineering for transcriptional control elements capable of increasing gene expression in plants. The inventors have now shown for the first time that the 5′ untranslated region (5′UTR), and particularly the first intron of a plant GOS2 gene, has a stimulating effect on transgene expression in plants when inserted between a promoter and a nucleic acid to be expressed. This was found not only to be the case when the first intron of a plant GOS2 gene was combined with the rice GOS2 promoter, but was surprisingly also observed when a plant GOS2 5′UTR, and in particular its first intron, was operably linked to a plant-expressible non-GOS2 promoter.

### SUMMARY OF THE INVENTION

The present invention provides a method for increasing transgene expression in a transgenic plant. The method comprises the steps of: (a) integrating all or a part of a 5′ UTR of a plant GOS2 gene in or at the 5′ end of a nucleic acid of interest, thereby creating a chimeric transcriptional unit, wherein said part of a 5′UTR comprises at least the first intron of said GOS2 gene or a functional variant of said first intron, which functional variant is at least 65 base pairs in length, and comprises splice sites and a functional branchpoint adenosine; (b) operably fusing said chimeric transcriptional unit to a plant-expressible promoter so as to obtain an expression cassette, provided there is no combination of a complete 5′ UTR from the rice GOS2 gene with a promoter of the rice GOS2 gene; (c) introducing into and expressing in a plant cell said expression cassette of (b), to create a transgenic plant cell; and (d) regenerating and/or growing a plant from said transgenic plant cell of (c) wherein said transgenic cell transcribes the transgene and wherein the 5′UTR of the expressed nucleic acid of interest comprises at least the 5′UTR as defined in (a).

Preferably, the functional variant is able to hybridise under stringent conditions to a naturally occurring first intron of a plant GOS2 gene. In another preferred embodiment, the transgene encodes a polypeptide.

In still another preferred embodiment, the transgenic plant is a monocotyledonous plant including but not limited to rice or maize. In yet another preferred embodiment, the GOS2 gene originates from a monocotyledonous plant, preferably from rice or maize.

Any plant-expressible promoter may be used in the methods and the compositions of the present invention. Examples include promoters such as constitutive promoters; inducible promoters; tissue-preferred promoters; organ-preferred promoters; and developmentally regulated promoters. An example of an organ-preferred promoter for use in the present invention is a seed-preferred promoter. An example of a tissue-preferred promoter for use in the present invention is an endosperm-preferred promoter.

The present invention also provides a method for increasing protein content of plant seeds. The method comprises the steps of:
a) integrating all or a part of a 5′ UTR of a plant GOS2 gene in or at the 5′ end of a nucleic acid encoding a protein of interest, thereby creating a chimeric transcriptional unit, wherein said part of a 5′UTR comprises at least the first intron of said GOS2 gene or a functional variant of said first intron, which functional variant is at least 65 base pairs in length, comprises splice sites and a functional branchpoint adenosine;
b) operably fusing said chimeric transcriptional unit to a plant-expressible promoter so as to obtain an expression cassette, provided there is no combination of a complete 5′ UTR from the rice GOS2 gene with a promoter of the rice GOS2 gene;
c) introducing into and expressing in a plant cell the expression cassette of (b) to create a transgenic plant cell; and
d) regenerating and/or growing a plant from the transgenic plant cell of (c) so that said transgenic plant cell transcribes said nucleic acid encoding the protein of interest and wherein the 5′UTR of the expressed nucleic acid encoding said protein comprises at least the 5′UTR of (a).

Preferably, the functional variant is able to hybridise under stringent conditions to a naturally occurring first intron of a plant GOS2 gene. In another preferred embodiment, the chimeric transcriptional unit of the present invention comprises a nucleic acid of interest fused at its 5′ end to the 3′ end of the 5′UTR of a GOS2 gene, or part thereof, wherein said part of a 5′UTR comprises at least the first intron of a GOS2 gene or a functional variant of said first intron.

The present invention also provides an isolated nucleic acid molecule comprising the 5′UTR of a plant GOS2 gene, or part of such 5′UTR, wherein said part of the GOS2 5′UTR comprises at least the first intron of said GOS2 gene or a functional variant of said first intron and provided there is no combination of a complete 5′ UTR from the rice GOS2 gene with a promoter of the rice GOS2 gene.

Examples of a 5′UTR of a GOS2 gene for use in the present invention include but are not limited to the sequences represented set forth in SEQ ID NO: 1 or SEQ ID NO:2.

### BRIEF DESCRIPTION OF THE DRAWINGS

FIG. 1 is a schematic representation of the rice GOS2 gene. The bottom arrow shows the structure of the mRNA, the white parts represent the introns, whereas the top arrow depicts the corresponding coding sequence (filled parts).

FIG. 2 is a schematic representation of the T-DNA of plasmid p5024, comprising starting from the left border (LB) a nptII expression cassette for resistance to kanamycin (SM k7) within 2 recombination sites (RS), the promoter of the rice prolamin RP6 gene, the cocksfoot mottle virus (CfMV) epsilon leader (enhancer, GenBank Z48630), the Gateway cassette A (GW), containing a chloramphenicol resistance gene and a ccdB suicide gene flanked by Gateway recombination site attR1 and affR2, a double terminator sequence (T-zein and T-rbcS) flanked by the right border (RB) sequence of the T-DNA.

FIG. 3 is a schematic representation of the T-DNA of plasmid p5942, derived from p5024 by inserting the first intron of rice GOS2 in the SpeI restriction site.

FIG. 4A is a schematic representation of the T-DNA of plasmid p06192 used for transforming rice plants. The GW cassette of p5024 is replaced by a reporter gene.

FIG. 4B is a schematic representation of the T-DNA of plasmid p06193 used for transforming rice plants. The GW cassette of p5942 is replaced by a reporter gene.

FIG. 5 (SEQ. ID NO:1) shows the nucleotide sequence for the part of the 5′UTR of rice GOS2 which is used in the examples. The intron sequence itself (SEQ ID NO: 5) is indicated in bold.

### DETAILED DESCRIPTION OF THE INVENTION

In one embodiment, the invention concerns a method for increasing transgene expression in a transgenic plant, which method comprises
a. integrating all or a part of a 5′ UTR of a plant GOS2 gene in or at the 5′ end of a nucleic acid of interest, thereby creating a chimeric transcriptional unit, wherein said part of a 5′UTR comprises at least the first intron of said GOS2 gene or a functional variant of said first intron;
b. operably fusing the chimeric transcriptional unit to a plant expressible promoter so as to obtain an expression cassette, provided there is no combination of a complete 5′ UTR from the rice GOS2 gene with a promoter of the rice GOS2 gene;
c. introducing into and expressing in a plant cell the expression cassette of (b), to create a transgenic plant cell; and
d. regenerating and/or growing a plant from the transgenic plant cell of (c) so that the transgenic cell transcribes the transgene and wherein the 5′UTR of the expressed nucleic acid of interest comprises at least the 5′UTR of (a).

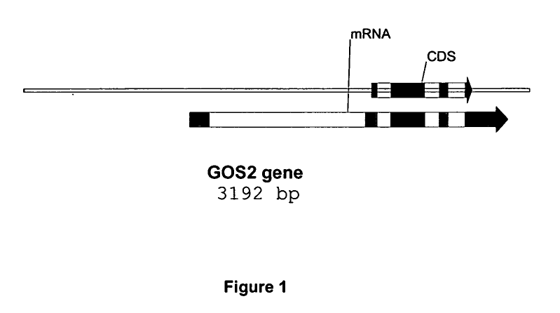

The subject of the present invention concerns the first intron of a GOS2 gene. The GOS2 gene (coding sequence given in SEQ ID NO: 3), also known as SUI1, encodes a translation initiation factor eIF1 (SEQ ID NO: 4). In a particular embodiment, the first intron of the rice GOS2 gene is used, but a person skilled in the art will appreciate that first introns of other GOS2 genes may also be used in the methods of the invention. The rice GOS2 gene contains four introns of respectively 998 bp, 94 bp, 90 bp and 105 bp (de Pater et al., Plant J. 2, 837-844, 1992). The first intron is located within the 5′UTR (FIG. 1), not only in rice (X51910), but also in Coffea arabica (AJ519840), and Arabidopsis (At5g54940, At5g54760, At1 g54290, At4g27130). It may be expected that this is the case for all plants, given the high sequence conservation among plant GOS2 genes. Therefore, in a preferred embodiment of this invention, alternative intron sequences of GOS2 genes are derived from plant GOS2 genes, because in these genes, the first introns are located in the 5′UTR. More preferably, the first intron of a GOS2 gene is derived from a monocotyledonous plant, most preferably from rice or maize. Nevertheless, a person skilled in the art would be able to isolate GOS2 first intron sequences from other plants without undesired flanking coding sequences by using standard techniques. Therefore, first introns derived from any plant GOS2/SUI1 gene may equally be useful in the methods of this invention.

The term “intron” or “intervening sequence” as defined herein is a non-coding nucleic acid sequence frequently interrupting coding sequences of eukaryotic genes but which may also be present outside the coding sequence and which are transcribed into RNA and subsequently removed by RNA splicing to leave a functional mRNA or other RNA. The intron comprises splice sites at both ends and a branchpoint adenosine. The term “splice sites” as defined herein is taken to mean the outer ends of the sequence that are spliced out from the primary transcript and the immediate nucleotides in the primary transcript of the sequence that is spliced out. The branchpoint adenosine is the nucleotide that forms the lariat during the event of splicing. The branchpoint adenosine is part of a conserved motif with the following consensus sequence (Brown, Plant Journal 10, 771-780, 1996): Y₁₀₀U₁₀₀R₆₄A₁₀₀U₅₀ or Y₁₀₀U₁₀₀R₆₄A₁₀₀Y₇₀, wherein Y stands for the nucleotides C or T, and R for A or G and wherein the subscript numbers denote the percentage of conservation of the respective nucleotide. This motif determines the functionality of the branchpoint adenosine.

Furthermore, functional variants of the first intron of a GOS2 gene may also be useful in increasing expression of a transgene. Functional variants encompass substitution, insertion and deletion variants and combinations thereof. “Substitution variants” are those in which at least one base in the nucleotide sequence has been removed and a different base inserted in its place. “Insertional variants” of a nucleic acid are those in which one or more nucleotides are introduced into a predetermined site in the sequence. “Deletion variants” of a nucleic acid are characterised by the removal of one or more nucleotides from the nucleic acid. Any combination of substitution(s), deletion(s) or insertion(s) may occur provided that the functionality of the intron remains essentially the same, that is, that the intron variant, when used in the methods according to the present invention, causes increased expression of a transgene. The increased expression is to be understood as increased when compared to expression of the same transgene without the presence of the intron. In addition, a functional variant is at least 65 base pairs in length, comprises splice sites and a functional branchpoint adenosine, and is able to hybridise under stringent conditions to a naturally occurring first intron of a plant GOS2 gene.

The term “hybridisation” as defined herein is a process wherein substantially homologous complementary nucleotide sequences anneal to each other. In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration and hybridisation buffer composition. “Stringent hybridisation conditions” and “stringent hybridisation wash conditions” in the context of nucleic acid hybridisation experiments such as Southern and Northern hybridisations are sequence dependent and are different under different environmental parameters. For example, longer sequences hybridise specifically at higher temperatures. The Tₘ is the temperature under defined ionic strength and pH, at which 50% of the target sequence hybridises to a perfectly matched probe. Specificity is typically the function of post-hybridisation washes. Critical factors of such washes include the ionic strength and temperature of the final wash solution. Generally, stringent conditions are selected to be about 50° C. lower than the thermal melting point (Tₘ) for the specific sequence at a defined ionic strength and pH. The Tₘ is the temperature under defined ionic strength and pH, at which 50% of the target sequence hybridises to a perfectly matched probe. The Tₘ is dependent upon the solution conditions and the base composition of the probe, and may be calculated using the following equation:
Tₘ=79.8° C.+(18.5×log[Na⁺])+(58.4° C.×%[G+C])−(820×(#bp in duplex)⁻¹)−(0.5×%formamide)

Preferred stringent conditions are when the temperature is 20° C. below Tₘ, and most preferred stringent conditions are when the temperature is 10° C. below Tₘ. Non-specific binding may also be controlled using any one of a number of known techniques such as blocking the membrane with protein containing solutions, additions of heterologous RNA, DNA, and SDS to the hybridisation buffer, and treatment with Rnase. Wash conditions are typically performed at or below hybridisation stringency. Generally, suitable stringent conditions for nucleic acid hybridisation assays or gene amplification detection procedures are as set forth above. Conditions of increased or decreased stringency compared to the above may also be selected.

For the purposes of defining the level of stringency, reference can conveniently be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989). The skilled artisan is aware of various parameters which may be altered during hybridisation and washing and which will either maintain or change the stringency conditions. An example of high stringency conditions includes hybridisation in 0.1-1×SSC/0.1% w/v SDS at 60° C. for 1-3 hours. Another example of stringent hybridisation conditions is hybridisation at 4×SSC at 65° C., followed by a washing in 0.1×SSC, at 65° C. for about one hour. Alternatively, an exemplary stringent hybridisation condition is in 50% formamide, 4×SSC at 42° C. Still another example of stringent conditions include hybridisation at 62° C. in 6×SSC, 0.05× BLOTTO and washing at 2×SSC, 0.1% w/v SDS at 62° C.

Plant GOS2 genes have their first intron located within the 5′UTR, therefore it is envisaged that it is not only this first intron but also the first intron with its flanking sequences up to the whole of the 5′UTR that may have a stimulating effect on gene expression. Therefore, the present invention provides in another embodiment a method for increasing transgene expression in a transgenic plant wherein substantially the complete 5′UTR of a plant GOS2 gene, or any part thereof comprising the first intron or a functional variant thereof, is used to increase expression of a transgene, provided that the rice GOS2 promoter is not used in combination with the complete 5′ UTR of the rice GOS2 gene.

The 5′UTR of a gene is to be understood as that part of a gene which is transcribed into a primary RNA transcript (pre-mRNA) and which part is located upstream of the coding sequence. The primary transcript is the initial RNA product, containing introns and exons, produced by transcription of DNA. Many primary transcripts must undergo RNA processing to form the physiologically active RNA species. The processing into a mature mRNA may comprise trimming of the ends, removal of introns, capping and/or cutting out of individual rRNA molecules from their precursor RNAs. The 5′UTR of an mRNA is thus that part of the mRNA which is not translated into protein and which is located upstream of the coding sequence.

According to the present invention, the 5′UTR of a GOS2 gene or a part thereof comprising the first intron, is integrated in or at the 5′ end of a nucleic acid of interest. The fusion results in the formation of a transcriptional unit, which gives rise to a single functional primary RNA transcript that can be processed to a mature mRNA in a given host cell. The 5′UTR of a GOS2 gene, or a part thereof comprising the first intron, is present in the transcriptional unit in sense orientation and upstream of any protein encoding sequence. The intron useful in the methods of the present invention is usually flanked by the border sequences which comprise the splice site, but may also be functional without these border sequences.

Preferably the nucleic acid of interest is a transgene. The term “nucleic acid sequence(s)”, “nucleic acid”, or “gene(s)”, when used herein refers to nucleotides, either ribonucleotides (RNA) or deoxyribonucleotides (DNA) or a combination of both, in a polymeric form of any length. These terms furthermore include double-stranded and single-stranded DNA and RNA. These terms also include nucleotide modifications known in the art, such as methylation, cyclisation, ‘caps’, substitution of one or more of the naturally occurring nucleotides with an analogue such as inosine, and polynucleotide backbone modifications. The term “transgene” refers to an isolated nucleic acid that is introduced into a plant. In a preferred embodiment of the present invention, the transgene encodes a polypeptide.

The term “polypeptide” or “protein” is defined as a molecular chain comprising two or more amino acids covalently joined by peptide bonds.

The term “isolated” in the present application means removed from its original environment. For example, a nucleic acid present in its natural state in an organism is not isolated, whereas the same nucleic acid separated from the adjacent nucleic acids in which it is naturally present, is regarded as being “isolated”. The transgene may originate from the same plant species or from another, it may be isolated from any source, such as bacteria, yeast or fungi, plants (including algae) or animals (including humans). The transgene may be substantially modified from its native form in composition and/or genomic environment through deliberate human manipulation. In a particular embodiment of the invention, the transgene comprises a sequence encoding a protein. However the transgene need not encode a protein, the methods may be applied for any nucleic acid to be expressed, such as tRNA, rRNA, anti-sense RNA, siRNA. A “coding sequence” or “open reading frame” (ORF) is defined as a nucleotide sequence that may be transcribed into mRNA and may later be translated into a polypeptide when placed under the control of appropriate regulatory sequences, i.e. when the coding sequence or ORF is present in an expressible form. The coding sequence or ORF is bound by a 5′ translation start codon and a 3′ translation stop codon. A coding sequence or ORF may include, but is not limited to RNA, mRNA, cDNA, recombinant nucleotide sequences, synthetically manufactured nucleotide sequences or genomic DNA. The coding sequence or ORF may be interrupted by introns.

Advantageously the methods of the present invention result in increased expression of the transgene. The term “expression” as defined herein is taken to mean the production of a protein or nucleotide sequence in a cell itself or in a cell-free system. It includes transcription into an RNA product, post-transcriptional modification and optionally also translation to a protein product or polypeptide from a DNA encoding that product, as well as possible post-translational modifications. A coding sequence may be transcribed in sense or antisense direction.

As mentioned above, any GOS2/SUI1 gene may be a suitable source of a 5′UTR and/or a first intron for use in a method for increasing transgene expression. Methods for the search and identification of eIF1 homologues would be well within the realm of persons skilled in the art. Such methods comprise comparison of the sequences represented by SEQ ID NO 3 or 4, in a computer readable format, with sequences that are available in public databases such as MIPS (http://mips.gsf.de/), GenBank (http://www.ncbi.nlm.nih.gov/Genbank/index.html) or EMBL Nucleotide Sequence Database (http://www.ebi.ac.uk/embl/index.html), using algorithms well known in the art for the alignment or comparison of sequences, such as GAP (Needleman and Wunsch, J. Mol. Biol. 48; 443453 (1970)), BESTFIT (using the local homology algorithm of Smith and Waterman (Advances in Applied Mathematics 2; 482-489 (1981))), BLAST (Altschul, S. F., Gish, W., Miller, W., Myers, E. W. & Lipman, D. J., J. Mol. Biol. 215:403-410 (1990)), FASTA and TFASTA (W. R. Pearson and D. J. Lipman Proc. Natl. Acad. Sci. USA 85:2444-2448 (1988)). The software for performing BLAST analysis is publicly available through the National Centre for Biotechnology Information. Suitable homologues may be identified using BLAST default parameters (BLOSUM62 matrix, gap opening penalty 11 and gap extension penalty 1). Preferably, full-length sequences (from start to stop codon in case of DNA, or of the complete mature protein) are used for analysis.

Analysis of genomic sequences for the identification of GOS2/SUI1 homologues is also possible. Several algorithms and software tools for the identification of genes in raw DNA sequence are available. Usually these tools combine analysis of statistical parameters in the DNA sequence with homology-based methods for identifying homologous sequences in databases. Although none of these methods alone is reliable enough for a good prediction, the combination of various programs usually gives good results. Well known examples of such tools include GeneMark (http://opal.biology.gatech.edu/genemark, http://www.ebi.ac.uk/genemark, Borodovsky, M. and McIninch J. (1993) GeneMark: Parallel Gene Recognition for both DNA Strands. Computers & Chemistry, 17, 123-133), Gene Locator and Interpolated Markov Modeler (GLIMMER, http://www.tigr.org/softlab, A. L. Delcher et al. Improved microbial gene identification with GLIMMER. Nucleic Acids Research, 27, 4636-4641. (1999)), Gene Recognition and Assembly Internet Link (GRAIL, http://compbio.ornl.gov), GenScan (http://genes.mit.edu/GENSCAN.html, Burge, C. and Karlin, S. (1997) Prediction of complete gene structures in human genomic DNA. J. Mol. Biol. 268, 78-94.), GeneBuilder (http://www.itba.mi.cnr.it/webgene Milanesi L. et al. GeneBuilder: interactive in silico prediction of genes structure. Bioinformatics, 15 (7):612-621, 1999). A combined analysis may be performed with the TIGR Combiner program (http://www.tigr.org/softlab, J. E. Allen et al. Computational gene prediction using multiple sources of evidence. Genome Research, 14(1), 142-148, 2004.) that predicts gene models using the output from other annotation software such as GeneMark, GlimmerM, GRAIL, GenScan, and Fgenes. It uses a statistical algorithm to identify patterns of evidence corresponding to gene models.

Methods for characterising the 5′UTR of a gene are known in the art. For example, many transcription starting points in eukaryotes are preceded by a TATA box or consist of an initiator element recognised by RNA polymerase 11. Naturally occurring initiator elements in many genes have a cytosine (C) at the −1 position and an adenine (A) residue at the transcription-start site (+1). The consensus sequence for an initiator element consists of 5′-Y Y A⁺¹ N T/A Y Y Y-3′, where A⁺¹ is the transcription start, Y is a pyrimidine (C or T), N is any of the four bases, and T/A is T or A at position +3. Other genes contain a typical CG-rich stretch of 20 to 50 nucleotides within a region of approximately 100 base pairs upstream of the start-site region, known as CpG island. Methods for predicting the presence of introns are also known in the art. Examples include SPL (http://www.softberry.com/berry.phtml, Softberry), NNSplice (http://www.fruitfly.org/seq tools/splice.html, Reese M G et al. 1997. Improved Splice Site Detection in Genie. J Comp Biol 4(3), 311-23), SpliceView (http://www.itba.mi.cnr.it/webgene/, Rogozin I. B. and L. Milanesi. Analysis of donor splice signals in different organisms. J. Mol. Evol., 1997, V.45, 50-59.), NetGene2 (http://www.cbs.dtu.dk/services, S. M. Hebsgaard, et al. Splice site prediction in Arabidopsis thaliana DNA by combining local and global sequence information, Nucleic Acids Research, 1996, Vol. 24, 3439-3452.), GeneSplicer (http://www.tigr.org/softlab, Pertea M. et al. GeneSplicer: a new computational method for splice site prediction. Nucleic Acids Res. 2001 Mar. 1; 29(5):1185-90).

In a preferred embodiment, the present invention provides a method for increasing expression of a transgene as outlined above, wherein said part of the GOS2 5′UTR is as represented by SEQ ID NO 1. In an alternative embodiment, the complete GOS2 5′UTR as represented by SEQ ID NO 2 is used. In any case, the methods according to the present invention always make use of a chimeric transcriptional unit comprising the nucleic acid of interest fused at its 5′ end to the 3′ end of the 5′UTR of a GOS2 gene, or part thereof, wherein that part of the GOS2 5′UTR comprises at least the first intron of the GOS2 gene or a functional variant of this first intron. Preferably, the part of the GOS2 5′UTR is as presented by SEQ ID NO 1.

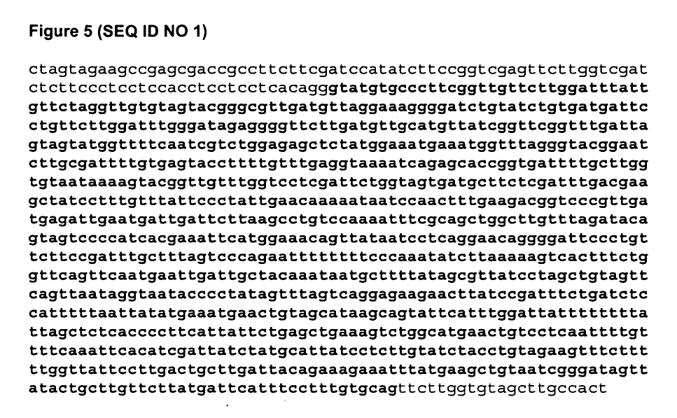

SEQ ID NO:1 is also set forth in FIG. 5. The intron sequence itself is bolded in FIG. 5 and also set forth in SEQ ID NO:5. SEQ ID NO 2 represents the complete 5′UTR of the rice GOS2 gene defined by de Pater et al. (1992), extracted from GenBank accession X51910. Transcription may start at any of the first three nucleotides. SEQ ID NO: 3 represents the coding sequence of the GOS2 translation initiation factor and SEQ ID NO: 4 is the GOS2 protein sequence encoded by SEQ ID NO: 3.

As is generally accepted, a nucleic acid strand is directional, with the “5′ end” having a free hydroxyl (or phosphate) on a 5′ carbon and the “3′ end” having a free hydroxyl (or phosphate) on a 3′ carbon. In case of a double stranded nucleic acid, the orientation of the sense strand establishes the direction. The sense strand has, by definition, the same ‘sense’ as the mRNA; that is, it may be translated exactly as the mRNA sequence can.

In a next step, the transcriptional unit is operably linked to a promoter, thereby forming an expression cassette. As used herein, a “promoter” means a region of DNA upstream from the transcription start and which is involved in binding RNA polymerase and other proteins to start transcription. Reference herein to a “promoter” is to be taken in its broadest context and includes the transcriptional regulatory sequences derived from a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. Consequently, a repressible promoter's rate of transcription decreases in response to a repressing agent. An inducible promoter's rate of transcription increases in response to an inducing agent. A constitutive promoter's rate of transcription is not specifically regulated, though it may vary under the influence of general metabolic conditions. The term “promoter” also includes the transcriptional regulatory sequences of a classical prokaryotic gene, in which case it may include a −35 box sequence and/or a −10 box transcriptional regulatory sequences. The term “promoter” is also used to describe a synthetic or fusion molecule, or derivative which confers, activates or enhances expression of a nucleic acid molecule in a cell, tissue or organ.

Advantageously, any type of promoter may be combined with the transcriptional unit and used to drive expression of the transgene, depending on the desired outcome. Advantageously, the promoter is a plant expressible promoter. By “plant expressible promoter” is meant a promoter which is capable of functioning in a plant cell. For example, a meristem-specific promoter, such as the rnr (ribonucleotide reductase), cdc2a promoter and the cyc07 promoter, may be used to effect expression in all growing parts of the plant, thereby increasing cell proliferation, which in turn would increase yield or biomass. If the desired outcome would be to influence seed characteristics, such as the storage capacity, seed size, seed number, biomass etc., then a seed-specific promoter, such as p2S2, pPROLAMIN, pOLEOSIN may be selected. An aleurone-specific promoter may be selected in order to increase growth at the moment of germination, thereby increasing the transport of sugars to the embryo. An inflorescence-specific promoter, such as pLEAFY, may be utilised if the desired outcome would be to modify the number of flower organs. To produce male-sterile plants one might want to use an anther specific promoter. To impact on flower architecture, for example petal size, one may choose a petal-specific promoter. If the desired outcome would be to modify growth and/or developmental characteristics in particular organs, the choice of the promoter to combine with would depend on the organ to be modified. For example, use of a root-specific promoter would lead to increased growth and/or increased biomass or yield of the root and/or phenotypic alteration of the root. This would be particularly important where it is the root itself that is the desired end product; such crops include sugar beet, turnip, carrot, and potato. A fruit-specific promoter may be used to modify, for example, the strength of the outer skin of the fruit or to increase the size of the fruit. A green tissue-specific promoter may be used to increase leaf size. A cell wall-specific promoter may be used to increase the rigidity of the cell wall, thereby increasing pathogen resistance. An anther-specific promoter may be used to produce male-sterile plants. A vascular-specific promoter may be used to increase transport from leaves to seeds. A nodule-specific promoter may be used to increase the nitrogen fixing capabilities of a plant, thereby increasing the nutrient levels in a plant. A stress-inducible promoter may also be used to drive expression of a nucleic acid to increase membrane integrity during conditions of stress. A stress-inducible promoter such as the water stress-inducible promoter WSI18, the drought stress-inducible Trg-31 promoter, the ABA related promoter rab21 or any other promoter which is inducible under a particular stress condition such as temperature stress (cold, freezing, heat) or osmotic stress, or drought stress or oxidative stress or biotic stress may be used to drive expression of a transgene.

In one particular embodiment of the invention, the promoter used for increasing the expression of a transgene is a constitutive promoter. The term “constitutive” as defined herein refers to a promoter expressed predominantly in at least one tissue or organ and predominantly at any life stage of the plant. Preferably the promoter is expressed predominantly throughout the plant. Preferably, the constitutive promoter is a GOS2 promoter, or a promoter of similar strength and/or a promoter with a similar expression pattern, more preferably the promoter is the GOS2 promoter from rice, provided that the rice GOS2 promoter is not used in combination with the complete 5′ UTR of the rice GOS2 gene. In another embodiment, an inducible promoter is used. An inducible promoter's rate of transcription increases in response to an inducing agent or condition, such as chemical, environmental, or physical stimuli. In still another embodiment, a developmentally regulated promoter is used. The term “developmentally regulated promoter” refers to a promoter whose activity is determined by developmental events. In a preferred embodiment, an organ-preferred promoter is used. An organ-preferred promoter is a promoter that is predominantly expressed in a specific organ of the plant. In a more preferred embodiment, a seed-preferred promoter is used. The term seed-preferred promoter refers to a promoter that is predominantly expressed in seeds of a plant. In another preferred embodiment, a tissue-preferred promoter is used. The term “tissue-preferred” as defined herein refers to a promoter that is expressed predominantly but not necessarily exclusively in one tissue or organ, but that may also be expressed in one specific cell. Preferably the tissue-preferred promoter is an endosperm-preferred promoter. Further preferably, the endosperm preferred promoter is a prolamin RP6 promoter, more preferably the prolamin RP6 promoter from rice.

The inventors have also shown for the first time that a 5′UTR originating from a plant GOS2 gene or a part thereof, and which part comprises the first intron, can be used to increase expression of a transgene in plant seeds.

Therefore, there is provided a method for increasing protein content of plant seeds, which method comprises
a. integrating all or a part of the 5′ UTR of a plant GOS2 gene in or at the 5′ end of a nucleic acid encoding the protein of interest, thereby creating a chimeric transcriptional unit, wherein this part of a GOS2 5′UTR comprises at least the first intron of the GOS2 gene or a functional variant of this first intron;
b. operably fusing the chimeric transcriptional unit to a plant expressible promoter so as to obtain an expression cassette, provided there is no combination of a complete 5′ UTR from the rice GOS2 gene with a promoter of the rice GOS2 gene;
c. introducing into and expressing in a plant cell the expression cassette of (b) to create a transgenic plant cell; and
d. regenerating and/or growing a plant from the transgenic plant cell of (c) so that the transgenic cell transcribes the nucleic acid encoding the protein of interest and wherein the 5′UTR of the expressed nucleic acid encoding the protein of interest comprises at least the 5′UTR of (a).

The term “operably linked” refers to a juxtaposition between a regulatory and a coding sequence, wherein the components so described are in a relationship permitting them to function in their intended manner. A regulatory sequence “operably linked” to a coding sequence is ligated in such a way that expression of the coding sequence is achieved under conditions compatible with the control sequences. In the case where the regulatory sequence is a promoter, it would be known to a skilled person that a double-stranded nucleic acid is preferable.

According to a further embodiment, there is provided a vector in which the expression cassette may be ligated. With “vector” or “genetic construct” is meant a DNA sequence, which may be introduced in an organism by transformation and may be transiently or stably maintained in this organism. Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences other than the first intron of a GOS2 gene, which may be suitable for use in performing the invention. Such sequences would be known or may readily be obtained by a person skilled in the art. The vector may further include an origin of replication sequence which is required for maintenance and/or replication in a specific cell type. One example is when a vector is required to be maintained in a bacterial cell as an episomal genetic element (e.g. plasmid or cosmid molecule). Preferred origins of replication include, but are not limited to, the f1-ori and colE1. The genetic construct may optionally comprise a selectable marker gene. As used herein, the term “selectable marker gene” includes any gene which confers a phenotype to a cell in which it is expressed to facilitate the identification and/or selection of cells which are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin); genes conferring resistance to herbicides (for example bar which provides resistance to Basta; aroA or gox providing resistance against glyphosate); or genes that provide a metabolic trait (such as manA that allows plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

In a next step, the expression cassette, whether or not inserted in a vector, is introduced into a plant cell or into the plant itself (including introduction into a tissue, organ or any other part of the plant). According to a preferred feature of the present invention, the nucleic acid is preferably introduced into a plant by transformation. The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome or into the chloroplast genome. The resulting transformed plant cell may then be used to regenerate a transformed plant in a manner known to persons skilled in the art.

Transformation of a plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., 1982, Nature 296, 72-74; Negrutiu I. et al., 1987, Plant Mol. Biol. 8, 363-373); electroporation of protoplasts (Shillito R. D. et al., 1985, Bio/Technol. 3, 1099-1102); microinjection into plant material (Crossway A. et al., 1986, Mol. Gen. Genet. 202, 179-185); DNA or RNA-coated particle bombardment (Klein T. M. et al., 1987, Nature 327, 70) infection with (non-integrative) viruses and the like. A preferred method for rice transformation according to the present invention is via Agrobacterium-mediated transformation using any of the well known methods for rice transformation, such as described in any of the following: published European patent application EP 1198985 A1, Aldemita and Hodges (Planta, 199, 612-617, 1996); Chan et al. (Plant Mol. Biol. 22 (3) 491-506, 1993), Hiei et al. (Plant J. 6 (2) 271-282, 1994), which disclosures are incorporated by reference herein as if fully set forth. In the case of corn transformation, the preferred method is as described in either Ishida et al. (Nat. Biotechnol. 1996 June; 14(6): 745-50) or Frame et al. (Plant Physiol. 2002 May; 129(1): 13-22), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis or by PCR, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

In a next step of selection, transformed plants are evaluated for the desired expression of the transgene or for the desired phenotypes. Methods for measuring modulated expression (increased or decreased expression) are known to the person skilled in the art. Altered RNA levels may be measured with techniques like RT-PCR whereas changes in protein levels may be determined by enzymatic assays, staining of SDS-PAGE protein gels, Western blotting, or enzyme-linked immunosorbent assays. Furthermore, in case of transgenic plants, changes in the phenotype of the transgenic plants may be monitored. For example, changes in yield or biomass, changes in plant architecture or stress response, or changes in protein or oil content of seeds may be measured. It is known to persons skilled in the art that the expression of transgenes in plants, and hence also the phenotypic effect due to expression of such transgene, may differ among different independently obtained transgenic lines and progeny thereof. The transgenes present in different independently obtained transgenic plants may differ from each other by the chromosomal insertion locus as well as by the number of transgene copies inserted, and by the configuration of those multiple transgene copies in a locus. Therefore differences in expression levels may be expected, which may be due to influence from the chromosomal context of the transgene (the so-called position effect) or from silencing mechanisms triggered by certain transgene configurations (e.g. inwards facing tandem insertions of transgenes are prone to silencing at the transcriptional or post-transcriptional level).

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to this invention. The invention also extends to harvestable parts of a plant, such as (but not limited to) seeds, leaves, fruits, flowers, stems or stem cultures, rhizomes, roots, tubers and bulbs. The invention further relates to products derived directly from a harvestable part of such a plant, such products including dry pellets or powders, oil, fat and fatty acids, starch or proteins.

The term “plant” as used herein encompasses whole plants, ancestors and progeny of the plants and plant parts, including seeds, fruits, flowers, leaves, shoots, stems, roots (including tubers), and plant cells, tissues and organs. The term “plant” therefore also encompasses suspension cultures, embryos, meristematic regions, callus tissue, gametophytes, sporophytes, pollen, and microspores. Plants that are particularly useful in the methods of the invention include algae, ferns, and all plants which belong to the superfamily Viridiplantae, in particular monocotyledonous and dicotyledonous plants, including fodder or forage legumes, ornamental plants, food crops, trees, or shrubs selected from the list comprising Abelmoschus spp., Acer spp., Actinidia spp., Agropyron spp., Allium spp., Amaranthus spp., Ananas comosus, Annona spp., Apium graveolens, Arabidopsis thaliana, Arachis spp, Artocarpus spp., Asparagus officinalis, Avena sativa, Averrhoa carambola, Benincasa hispida, Bertholletia excelsea, Beta vulgaris, Brassica spp., Cadaba farinosa, Camellia sinensis, Canna indica, Capsicum spp., Carica papaya, Carissa macrocarpa, Carthamus tinctorius, Carya spp., Castanea spp., Cichorium endivia, Cinnamomum spp., Citrullus lanatus, Citrus spp., Cocos spp., Coffea spp., Cola spp., Colocasia esculenta, Corylus spp., Crataegus spp., Cucumis spp., Cucurbita spp., Cynara spp., Daucus carota, Desmodium spp., Dimocarpus longan, Dioscorea spp., Diospyros spp., Echinochloa spp., Eleusine coracana, Eriobotrya japonica, Eugenia uniflora, Fagopyrum spp., Fagus spp., Ficus carica, Fortunella spp., Fragaria spp., Ginkgo biloba, Glycine spp., Gossypium hirsutum, Helianthus spp., Hibiscus spp., Hordeum spp., Ipomoea batatas, Juglans spp., Lactuca sativa, Lathyrus spp., Lemna spp., Lens culinaris, Linum usitatissimum, Litchi chinensis, Lotus spp., Luffa acutangula, Lupinus spp., Macrotyloma spp., Malpighia emarginata, Malus spp., Mammea americana, Mangifera indica, Manihot spp., Manilkara zapota, Medicago sativa, Melilotus spp., Mentha spp., Momordica spp., Morus nigra, Musa spp., Nicotiana spp., Olea spp., Opuntia spp., Omithopus spp., Oryza spp., Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Rubus spp., Saccharum spp., Sambucus spp., Secale cereale, Sesamum spp., Solanum spp., Sorghum bicolor, Spinacia spp., Syzygium spp., Tamarindus indica, Theobroma cacao, Trifolium spp., Triticosecale rimpaui, Triticum spp., Vaccinium spp., Vicia spp., Vigna spp., Vitis spp., Zea mays, Zizania palustris, Ziziphus spp., amongst others.

According to a preferred feature of the present invention, the plant is a crop plant comprising soybean, sunflower, canola, alfalfa, rapeseed or cotton. Further preferably, the plant according to the present invention is a monocotyledonous plant, including members of the Poaceae, such as sugarcane; most preferably a cereal, such as rice, maize, wheat, millet, barley, oats, sorghum.

The present invention also relates to use of the 5′UTR of a plant GOS2 gene or part thereof comprising the first intron. Any use of the 5′UTR of a plant GOS2 gene or part thereof comprising the first intron, for increasing transgene expression is envisaged, provided there is no combination of a complete 5′ UTR from the rice GOS2 gene with a promoter of the rice GOS2 gene. In particular, the 5′UTR of a plant GOS2 gene or part thereof comprising the first intron may be used for modifying the growth characteristics of a plant, such as yield, amount of produced biomass, architecture, stress tolerance. Other examples of use of the 5′UTR of a plant GOS2 gene or part thereof comprising the first intron include modification of the composition of a plant or harvestable parts thereof, such as seeds. Such use may result for example in seeds with increased protein content. Increased protein content of seeds is advantageous for increasing the nutritional value of these seeds or is furthermore advantageous in the field of molecular farming. Plants with improved growth characteristics may also be used to produce industrial proteins and/or other compounds. In this case, the goal would be to ensure high accumulation of the desired products in particular and easy-to-harvest plant tissues.

The present invention also relates to a composition, e.g., an isolated nucleic acid molecule comprising the 5′UTR of a plant GOS2 gene, or part thereof, for increasing expression of a transgene in transgenic plants provided that (i) said part of the GOS2 5′UTR comprises at least the first intron of said GOS2 gene or a functional variant of said first intron and (ii) the rice GOS2 intron is not used in combination with the rice GOS2 promoter.

The compositions of the present invention may be used in order to modify growth characteristics in a plant, increase yield in a plant, increase stress tolerance in a plant and alter architecture of a plant.

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### EXAMPLE 1

### Genetic Constructs

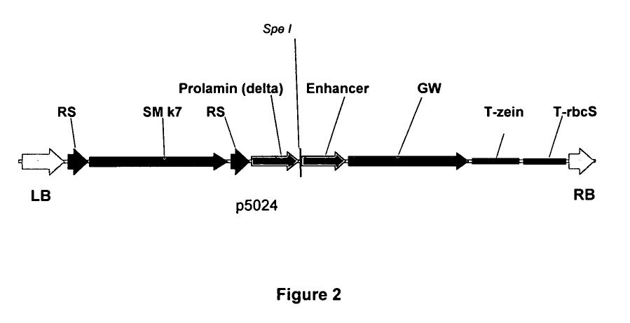

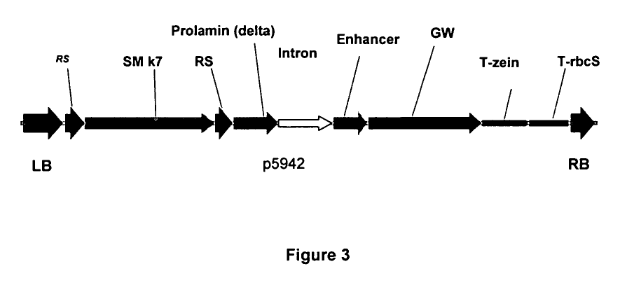

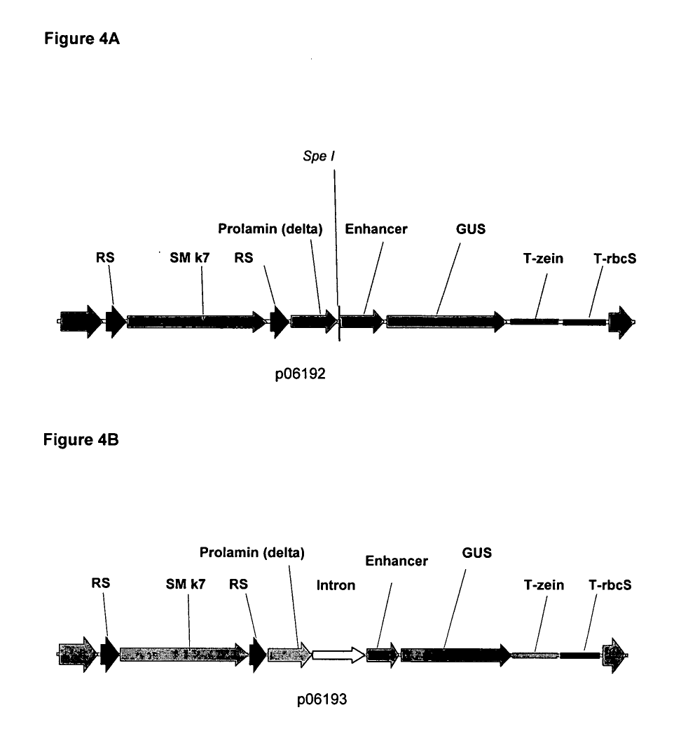

A modified T-DNA (p5024) was created comprising within its borders the elements as shown in FIG. 2. Next the first intron of the rice GOS2 gene was inserted in the restriction site SpeI (FIG. 3). Finally, the GW cassette was replaced by the GUS gene using a GW LR reaction, resulting in the plasmids p06192 and p06193 (FIG. 4).

### EXAMPLE 2

### Rice Transformation

Mature dry seeds of Oryza sativa japonica cultivar Nipponbare were dehusked. Sterilization was done by incubating the seeds for one minute in 70% ethanol, followed by 30 minutes in 0.2% HgCl₂ and by 6 washes of 15 minutes with sterile distilled water. The sterile seeds were then germinated on a medium containing 2,4-D (callus induction medium). After a 4-week incubation in the dark, embryogenic, scutellum-derived calli were excised and propagated on the same medium. Two weeks later, the calli were multiplied or propagated by subculture on the same medium for another 2 weeks. Three days before co-cultivation, embryogenic callus pieces were sub-cultured on fresh medium to boost cell division activity. The Agrobacterium strain LBA4404 harbouring the binary vector p06192 or p06193 was used for co-cultivation. The Agrobacterium strain was cultured for 3 days at 28° C. on AB medium with the appropriate antibiotics. The bacteria were then collected and suspended in liquid co-cultivation medium at an OD₆₀₀ of about 1. The suspension was transferred to a petri dish and the calli were immersed in the suspension for 15 minutes. The callus tissues were then blotted dry on a filter paper, transferred to solidified co-cultivation medium and incubated for 3 days in the dark at 25° C. Thereafter, co-cultivated callus was grown on 2,4-D-containing medium for 4 weeks in the dark at 28° C. in the presence of a selective agent at a suitable concentration. During this period, rapidly growing resistant callus islands developed. Upon transfer of this material to a regeneration medium, and incubation in the light, the embryogenic potential was released and shoots developed in the next four to five weeks. Shoots were excised from the callus and incubated for 2 to 3 weeks on an auxin-containing medium from which they were transferred to soil. Hardened shoots were grown under high humidity and short days in a greenhouse. Finally seeds were harvested three to five months after transplanting. The method yielded single locus transformants at a rate of over 50% (Aldemita and Hodges, Planta 199, 612-617, 1996; Chan et al., Plant Mol. Biol. 22(3), 491-506, 1993; Hiei et al., Plant J. 6(2), 271-282, 1994).

### EXAMPLE 3

### Evaluation of the Transformed Plants

Approximately 15 to 20 independent T0 transformants were generated. The primary transformants were transferred from tissue culture chambers to a greenhouse for growing and harvest of T1 seed. 10 and 15 events (respectively without and with intron) were retained. For each event, 10 T1 seeds (a 1:2:1 mix of nullizygotes, hemizygotes and homozygotes) were ground to a fine powder and assayed for GUS activity (modified from Breyne P, et al. (1993), Plant Mol. Biol. Reporter 11, 21-31) in triplicate measurements. Results are given in Table 1 below:

TABLE 1
Measurement of GUS activities in plants transformed
with construct with or without GOS2 intron:

| | | | | |
|---|---|---|---|---|
| Line | 1 | 2 | 3 | Average |

Promoter + Enhancer + GUS

| | | | | |
|---|---|---|---|---|
| OS1861-001A | 14.3 | 28.7 | 17.3 | 20 |
| OS1861-001B | 8.8 | 11.1 | 11.4 | 10 |
| OS1861-002A | 46.8 | 54.1 | 14.8 | 39 |
| OS1861-004A | 28.4 | 14.7 | 24.7 | 23 |
| OS1861-005C | 7.5 | 17.3 | 29.8 | 18 |
| OS1861-006A | 13.4 | 53.2 | 14.5 | 27 |
| OS1861-006B | 4.1 | 15.5 | 26.3 | 15 |
| OS1861-007B | 2.5 | 106.4 | NA | 54 |
| OS1861-009A | 18.3 | 118.7 | 92.4 | 76 |
| OS1861-009C | 13.5 | 11.7 | 1.9 | 9 |
| | | | average | 29 |
| | | | stdev | 22 |

Promoter + GOS2-intron + Enhancer + GUS

| | | | | |
|---|---|---|---|---|
| OS1866-001C | 67.1 | 109.5 | 80.4 | 86 |
| OS1866-002C | 40.6 | 153.2 | 259.8 | 151 |
| OS1866-003C | 158.5 | 129.6 | 200.1 | 163 |
| OS1866-005A | 173.8 | 302.8 | 61.9 | 180 |
| OS1866-005B | 162.1 | 217.1 | 65.6 | 148 |
| OS1866-007A | 27.4 | 221.2 | 281.7 | 177 |
| OS1866-007C | 72.4 | 130.2 | 183.8 | 129 |
| OS1866-008A | 196.6 | 186.2 | 117 | 167 |
| OS1866-008C | 161.5 | 336.6 | 61.2 | 186 |
| OS1866-011A | 162.0 | 238.1 | 403.4 | 268 |
| OS1866-012A | 118.7 | 115.4 | 449.8 | 228 |
| OS1866-013A | 33.6 | 55.4 | NA | 45 |
| OS1866-014A | 17.7 | 99.8 | 79 | 66 |
| OS1866-014B | 93.7 | 81.2 | 105.2 | 93 |
| OS1866-015A | 159.4 | 90.0 | 10.8 | 87 |
| | | | average | 149 |
| | | | stdev | 62 |

From the data it can be deduced that the presence of the GOS2 intron results in a more than 5-fold increase in expression of the reporter gene.

1. A method for increasing transgene expression in a transgenic plant, said method comprising:
a) integrating all or a part of a 5′ UTR of a plant GOS2 gene in or at the 5′ end of a nucleic acid of interest, thereby creating a chimeric transcriptional unit, wherein said part of a 5′UTR comprises at least the first intron of said GOS2 gene or a functional variant of said first intron, which functional variant is at least 65 base pairs in length, comprises splice sites and a functional branchpoint adenosine;
b) operably fusing said chimeric transcriptional unit to a plant-expressible promoter so as to obtain an expression cassette, provided there is no combination of a complete 5′ UTR from the rice GOS2 gene with a promoter of the rice GOS2 gene;
c) introducing into and expressing in a plant cell said expression cassette of (b), to create a transgenic plant cell; and
d) regenerating and/or growing a plant from said transgenic plant cell of (c) wherein said transgenic cell transcribes the transgene and wherein the 5′UTR of the expressed nucleic acid of interest comprises at least the 5′UTR as defined in (a).

2. The method of claim 1, wherein said functional variant is able to hybridise under stringent conditions to a naturally occurring first intron of a plant GOS2 gene.

3. The method of claim 1 or 2, wherein said transgene encodes a polypeptide.

4. The method of claim 1 or 2, wherein said transgenic plant is a monocotyledonous plant.

5. The method of claim 4, wherein said monocotyledonous plant is a cereal, such as rice or maize.

6. The method of claim 1 or 2, wherein said plant GOS2 gene originates from a monocotyledonous plant, preferably from rice or maize.

7. The method of claim 1 or 2, wherein said plant-expressible promoter is selected from the group consisting of: a constitutive promoter; an inducible promoter; a tissue-preferred promoter; an organ-preferred promoter; and a developmentally regulated promoter.

8. The method of claim 7, wherein said organ-preferred promoter is a seed-preferred promoter.

9. The method of claim 7, wherein said tissue-preferred promoter is an endosperm-preferred promoter.

10. The method of claim 1 or 2, wherein said 5′ UTR of a plant GOS2 gene is as represented by SEQ ID NO: 2.

11. The method according to claim 1 or 2, wherein said part of a 5′UTR of a plant GOS2 gene is as represented by SEQ ID NO: 1.

12. A method for increasing protein content of plant seeds, said method comprising:
a) integrating all or a part of a 5′ UTR of a plant GOS2 gene in or at the 5′ end of a nucleic acid encoding a protein of interest, thereby creating a chimeric transcriptional unit, wherein said part of a 5′UTR comprises at least the first intron of said GOS2 gene or a functional variant of said first intron, which functional variant is at least 65 base pairs in length, comprises splice sites and a functional branchpoint adenosine;
b) operably fusing said chimeric transcriptional unit to a plant-expressible promoter so as to obtain an expression cassette, provided there is no combination of a complete 5′ UTR from the rice GOS2 gene with a promoter of the rice GOS2 gene;
c) introducing into and expressing in a plant cell the expression cassette of (b) to create a transgenic plant cell; and
d) regenerating and/or growing a plant from the transgenic plant cell of (c) so that said transgenic plant cell transcribes said nucleic acid encoding the protein of interest and wherein the 5′UTR of the expressed nucleic acid encoding said protein comprises at least the 5′UTR of (a).

13. The method of claim 12, wherein said functional variant is able to hybridise under stringent conditions to a naturally occurring first intron of a plant GOS2 gene.

14. The method according to claim 1 or 12, wherein said chimeric transcriptional unit comprises a nucleic acid of interest fused at its 5′ end to the 3′ end of the 5′UTR of a GOS2 gene, or part thereof, wherein said part of a 5′UTR comprises at least the first intron of a GOS2 gene or a functional variant of said first intron.

15. The method of claim 12, wherein said part of the 5′UTR of a GOS2 gene is as represented by SEQ ID NO: 1.

16. An isolated nucleic acid molecule comprising the 5′UTR of a plant GOS2 gene, or part of said 5′UTR, wherein said part of the GOS2 5′UTR comprises at least the first intron of said GOS2 gene or a functional variant of said first intron and provided there is no combination of a complete 5′ UTR from the rice GOS2 gene with a promoter of the rice GOS2 gene.


SEQUENCE LISTING (5 sequences)

SEQ ID NO: 1 - DNA, 1112 bp, Oryza sativa
ctagtagaagccgagcgaccgccttcttcgatccatatcttccggtcgagttcttggtcgatctcttccctcctccacctcctcctcacagggtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgagtaccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccact

SEQ ID NO: 2 - DNA, 1176 bp, Oryza sativa
taggaggcatccaagccaagaagagggagagcaccaaggacacgcgactagcagaagccgagcgaccgccttcttcgatccatatcttccggtcgagttcttggtcgatctcttccctcctccacctcctcctcacagggtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgagtaccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatcgattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagtttc

SEQ ID NO: 3 - DNA, 348 bp, Oryza sativa
atgtctgatctcgacattcagatcccaactgccttcgatccctttgctgaggccaatgctggagactctggtgcggctgcaggatcaaaggactacgttcatgtacgcatccagcagcgtaatggccgtaagagcctgaccactgtccagggattgaagaaggaattcagctacaacaagatccttaaagatctcaagaaagagttttgctgcaatggtactgttgtccaggacccagagcttggccaggtcattcaacttcagggtgatcagaggaagaacgtatcaaattttcttgttcaggccggcattgtgaagaaggaacacatcaagattcatggtttctga

SEQ ID NO: 4 - protein, 115 aa, Oryza sativa
MSDLDIQIPTAFDPFAEANAGDSGAAAGSKDYVHVRIQQRNGRKSLTTVQGLKKEFSYNKILKDLKKEFCCNGTVVQDPELGQVIQLQGDQRKNVSNFLVQAGIVKKEHIKIHGF

SEQ ID NO: 5 - DNA, 999 bp, Oryza sativa
gtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgagtaccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcag